(19) United States
(12) Patent Application Publication
(10) Pub. No.: US 2022/0208312 A1
(43) Pub. Date: Jun. 30, 2022

(54) AUGMENTED REALITY LEWIS STRUCTURE EXPLORER

(71) Applicants: JULIA ENGLISH WINTER, BLOOMFIELD HILLS, MI (US); JOSEPH ENGALAN, ROYAL OAK, MI (US)

(72) Inventors: JULIA ENGLISH WINTER, BLOOMFIELD HILLS, MI (US); JOSEPH ENGALAN, ROYAL OAK, MI (US)

(21) Appl. No.: 17/563,297

(22) Filed: Dec. 28, 2021

Related U.S. Application Data
(60) Provisional application No. 63/131,137, filed on Dec. 28, 2020.

(51) Int. Cl. G16C 20/80; G09B 23/24; G09B 21/00; G06V 20/20

(52) U.S. Cl. CPC G16C 20/80; G06V 20/20; G09B 21/006; G09B 23/24

(57) ABSTRACT

An augmented reality Lewis structure explorer including a set of chemical pieces to be used to form representations of chemical structures is provided. The set of chemical pieces includes a first plurality of atom pieces and a second plurality of chemical bond pieces. Each atom piece represents an atom, while each chemical bond piece represents a chemical bond. The Lewis structure explorer also includes one or more video cameras configured to view a target area, a display, and a computer device in communication with the one or more video cameras and the display. The computer device is configured to recognize atom pieces and chemical bond pieces placed within the target area. The computer device is also configured to recognize chemical bonds when a selected chemical bond piece is placed between two atom pieces. The computer device further configured to render recognized atom pieces and chemical bond pieces on the display.

### CROSS-REFERENCE TO RELATED APPLICATIONS

This application claims the benefit of U.S. provisional application Ser. No. 63/131137 filed Dec. 28, 2020, the disclosure of which is hereby incorporated in its entirety by reference herein.

### TECHNICAL FIELD

In at least one aspect, an augmented reality system for creating and exploring chemical structures is provided.

### BACKGROUND

As new technologies revolutionize educational content delivery, with textbook publishers shifting to completely digital experiences, further marginalization of blind or visually impaired (BVI) students will occur if these new digital platforms do not provide accessible experiences for all students. This problem is especially magnified in STEM learning, due to the visual and spatial nature of the curricula, so BVI students are even less likely to pursue careers in the sciences. The central challenge is also an incredible opportunity: with this transformation to digital learning content we can re-imagine the possibilities and build assistive technology into the digital learning tools as a key feature as opposed to an after-thought.

Accordingly, there is a need for new technologies for teaching chemistry and delivering chemical content to students, and in particular to students that are blind or visually impaired.

### SUMMARY

In at least one aspect, an augmented reality Lewis structure explorer including a set of chemical pieces to be used to form representations of chemical structures is provided. The set of chemical pieces includes a first plurality of atom pieces and a second plurality of chemical bond pieces. Each atom piece represents an atom, while each chemical bond piece represents a chemical bond. The Lewis structure explorer also includes one or more video cameras configured to view a target area, a display, and a computer device in communication with the one or more video cameras and the display. The computer device is configured to recognize atom pieces and chemical bond pieces placed within the target area. The computer device is also configured to recognize chemical bonds when a selected chemical bond piece is placed between two atom pieces. The computer device further configured to render recognized atom pieces and chemical bond pieces on the display.

In another aspect, a chemical structure and/or chemical mechanism system is provided. The system includes a computer device configured to execute a chemical structure and/or chemical mechanism and an augmented reality Lewis structure explorer configured as an input device for the chemical structure and/or a chemical mechanism program. The augmented reality Lewis structure explorer including a set of chemical pieces to be used to form representations of chemical structures is provided. The set of chemical pieces includes a first plurality of atom pieces and a second plurality of chemical bond pieces. Each atom piece represents an atom, while each chemical bond piece represents a chemical bond. The Lewis structure explorer also includes one or more video cameras configured to view a target area, and a display. The computer device is in communication with the one or more video cameras and the display. The computer device is configured to recognize atom pieces and chemical bond pieces placed within the target area. The computer device is also configured to recognize chemical bonds when a selected chemical bond piece is placed between two atom pieces. The computer device further configured to render recognized atom pieces and chemical bond pieces on the display.

In another aspect, a software application that creates an Augmented Reality (AR) interface provides personalized formative assessment to users as they interact with physical objects to explore STEM concepts.

In another aspect, the AR physical model Explorer allows analysis of the move-by-move data from a completely digital interactive explorer.

In another aspect, the AR software interface provides dynamic and personalized assessment and audio feedback to students as they use the physical model system set.

Advantageously, the augmented reality Lewis structure explorer provides a learning tool that is accessible to BVI users.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1A. Schematic of an augmented reality Lewis structure explorer.

FIG. 1B. Schematic of a set of chemical pieces.

FIG. 2A. Schematic of the top side of an atom piece.

FIG. 2B. Schematic of the bottom side of an atom piece.

FIG. 3A. Schematic of the top side of a chemical bond piece.

FIG. 3B. Schematic of the bottom side of a chemical bond piece.

FIG. 4. Schematic of a molecule formed using the augmented reality Lewis structure explorer.

FIG. 5. Schematic of networked computer devices implementing the augmented reality Lewis structure explorer.

FIG. 6. Schematic of a computer device used in the augmented reality Lewis structure explorer.

### DETAILED DESCRIPTION

Reference will now be made in detail to presently preferred embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4. . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits.

For any device described herein, linear dimensions and angles can be constructed with plus or minus 50 percent of the values indicated rounded to or truncated to two significant figures of the value provided in the examples. In a refinement, linear dimensions and angles can be constructed with plus or minus 30 percent of the values indicated rounded to or truncated to two significant figures of the value provided in the examples. In another refinement, linear dimensions and angles can be constructed with plus or minus 10 percent of the values indicated rounded to or truncated to two significant figures of the value provided in the examples.

The term “connected to” means that the electrical components referred to as connected to are in electrical communication. In a refinement, “connected to” means that the electrical components referred to as connected to are directly wired to each other. In another refinement, “connected to” means that the electrical components communicate wirelessly or by a combination of wired and wirelessly connected components. In another refinement, “connected to” means that one or more additional electrical components are interposed between the electrical components referred to as connected to with an electrical signal from an originating component being processed (e.g., filtered, amplified, modulated, rectified, attenuated, summed, subtracted, etc.) before being received to the component connected thereto.

The term “electrical communication” means that an electrical signal is either directly or indirectly sent from an originating electronic device to a receiving electrical device. Indirect electrical communication can involve processing of the electrical signal, including but not limited to, filtering of the signal, amplification of the signal, rectification of the signal, modulation of the signal, attenuation of the signal, adding of the signal with another signal, subtracting the signal from another signal, subtracting another signal from the signal, and the like. Electrical communication can be accomplished with wired components, wirelessly connected components, or a combination thereof

The term “one or more” means “at least one” and the term “at least one” means “one or more.” The terms “one or more” and “at least one” include “plurality” as a subset.

The term “substantially,” “generally,” or “about” may be used herein to describe disclosed or claimed embodiments. The term “substantially” may modify a value or relative characteristic disclosed or claimed in the present disclosure. In such instances, “substantially” may signify that the value or relative characteristic it modifies is within ±0%, 0.1%, 0.5%, 1%, 2%, 3%, 4%, 5% or 10% of the value or relative characteristic.

The term “computer device” refers generally to any device that can perform at least one function, including communicating with another computer device. Examples of computer devices include bust are not limited to, smartphones, laptop computers, desktop computers, tablets (e.g., iPad), servers, and the like. Sometimes a computer device is referred to as a computer. Sometimes, a computer device is referred to as a computing device. Therefore, the terms “computing device” and “computer device” are interchangeable.

The processes, methods, or algorithms disclosed herein can be deliverable to/implemented by a processing device, controller, or computer, which can include any existing programmable electronic control unit or dedicated electronic control unit. Similarly, the processes, methods, or algorithms can be stored as data and instructions executable by a controller or computer in many forms including, but not limited to, information permanently stored on non-writable storage media such as ROM devices and information alterably stored on writeable storage media such as floppy disks, magnetic tapes, CDs, RAM devices, and other magnetic and optical media. The processes, methods, or algorithms can also be implemented in a software executable object. Alternatively, the processes, methods, or algorithms can be embodied in whole or in part using suitable hardware components, such as Application Specific Integrated Circuits (ASICs), Field-Programmable Gate Arrays (FPGAs), state machines, controllers or other hardware components or devices, or a combination of hardware, software and firmware components.

Throughout this application, where publications are referenced, the disclosures of these publications in their entireties are hereby incorporated by reference into this application to more fully describe the state of the art to which this invention pertains.

Abbreviations:

“AR” means augmented reality.

“BVI” means blind or visually impaired.

“STEM” means science, technology, engineering, and mathematics.

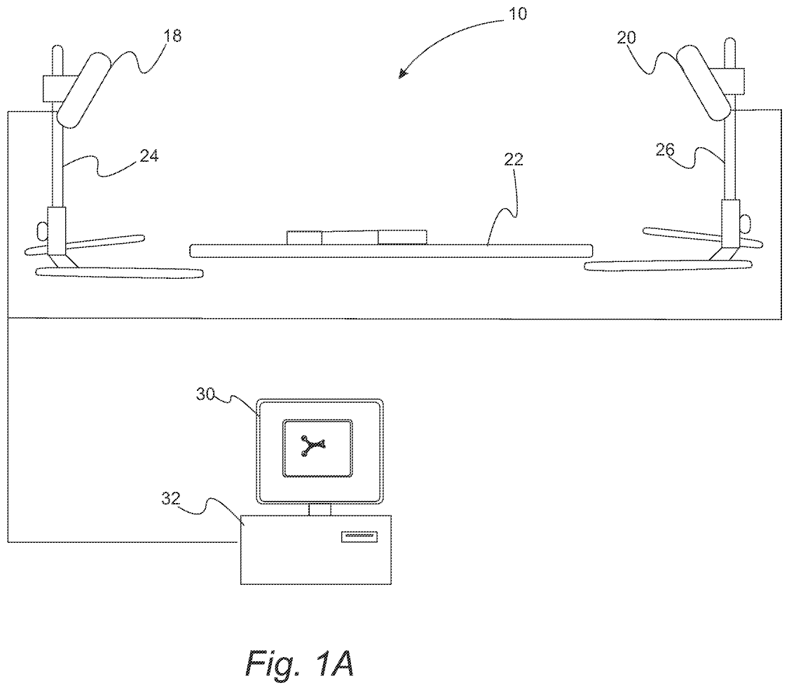

FIG. 1A provides a schematic of an augmented reality Lewis structure explorer. FIG. 1B provides a schematic of a set of chemical pieces. Augmented reality Lewis structure explorer 10 (i.e., a system) includes a set of chemical pieces 12 to be used to form representations of chemical structures. The set of chemical pieces 12 includes a first plurality of atom pieces 14 and a second plurality of chemical bond pieces 16. Each atom piece represents an atom. FIG. 1B provides as examples, hydrogen atom piece 14¹, carbon atom piece 14², oxygen atom piece 14³, chlorine atom piece 14⁴, and nitrogen atom piece 14⁵. Clearly, the plurality of atom pieces can include atom pieces for each atom in the Periodic Table. Similarly, each chemical bond piece represents a chemical bond. FIG. 1B provides as examples, single bond piece 16¹ representing a single bond, double bond piece 16² representing a double bond, and triple bond piece 16³ representing a triple bond. Augmented reality Lewis structure explorer 10 also includes one or more video cameras 18, 20 configured to view a target area 22. Video cameras 18, 20 are held by camera mounts 24, 26. In a refinement, the video cameras are webcams.

In a refinement, augmented reality Lewis structure Explorer 10 also includes a display 30 and a computer device 32 in communication with the one or more video cameras 18, 20 and the display 30. Computer device 32 is configured to each member of the set of chemical pieces 12 placed within the target area 22. Computer device 32 is configured to recognize atom pieces and chemical bond pieces placed within the target area 22. Computer device 32 is also configured to recognize chemical bonds when a selected chemical bond piece is placed between two atom pieces. Typically, a chemical bond piece will physically contact two atom pieces when forming a representation of a chemical bond. Computer device 32 is further configured to render recognized atom pieces and chemical bond pieces on display 30.

In a refinement, set of chemical pieces 12 includes further comprising positive charge 34 pieces representing positive charges and negative charge pieces 36 representing negative charges. In a further refinement, set of chemical pieces 12 further includes electron pair pieces 38 representing a lone pair of electrons. Set of chemical pieces 12 can also include single electron pieces for representing a single electron for forming a radical.

In a variation, the set of chemical pieces further includes a hint token 48 that provides feedback regarding the number of valence electrons and/or formal charge when the hint token 48 is positioned proximate to an atom piece or a chemical bond piece. For example, the feedback can includes an indication of missing parts of a chemical bond or octet. In a refinement, the feedback is audio feedback and, in particular, verbal feedback.

In a variation, the set of chemical pieces 12 further includes an arrow piece 49 that can be used to construct representations of chemical reactions.

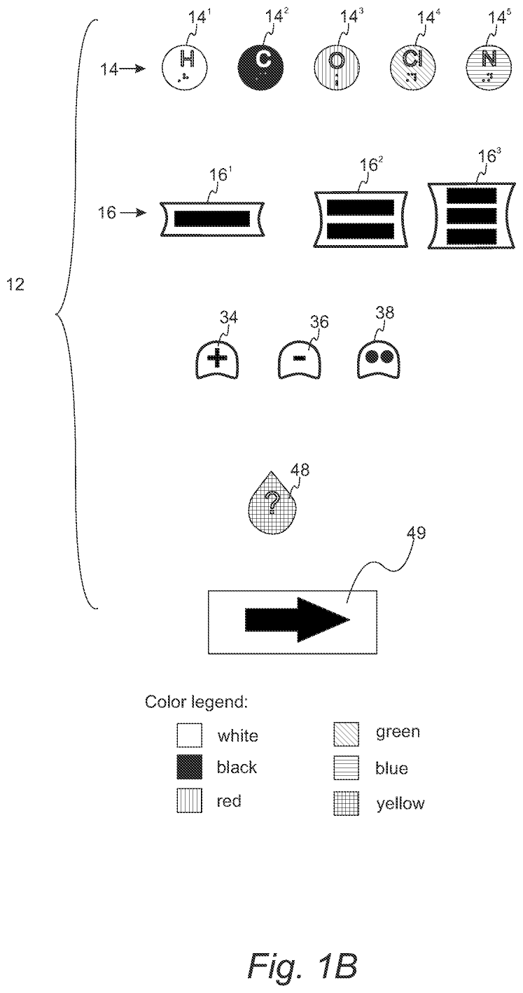

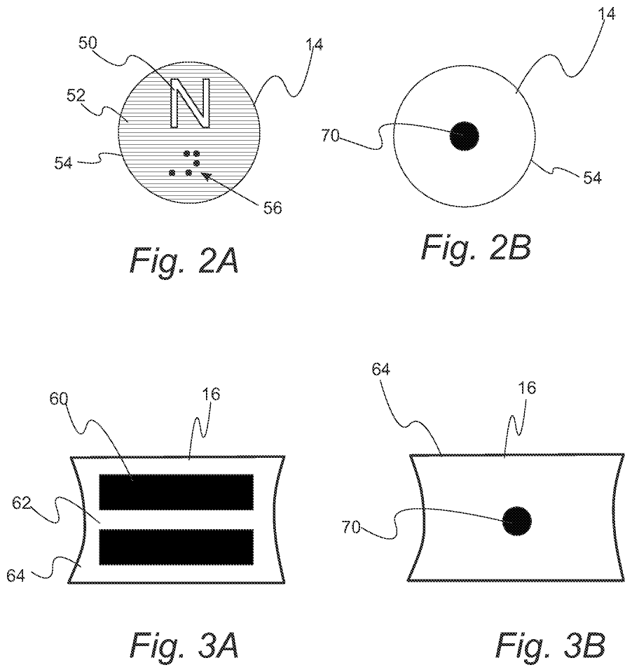

With reference to FIGS. 2A, 2B, 3A, and 3B, the construction of the pieces in the set of chemical pieces is illustrated. Typically, each piece is composed of a plastic base section with an optional embossment extending from a surface of the base section. As depicted in FIGS. 2A and 2B, atom piece 14 can include a letter(s) 50 extending from top surface 52 of disc-shaped base 54. The letters can be the chemical symbol for the atom. Therefore, letters and other chemical symbols can be the embossment (e.g., embossed chemical letter symbols). In addition, the letters for atoms and other symbols can be printed on the top face of the relevant piece. In a refinement, each atom piece can include a braille representation 56 (e.g., the chemical symbol) of the atom. Similarly, as depicted in FIGS. 3A and 3B, each chemical bond piece 16 can include bars 60 (e.g., embossments) extending from top surface 62 of the elongated base section 64 (e.g., one bar for a single bond, two bars for a double bond, and three bars for a triple bond). The ends of elongated base section 64 are curved and shaped to conform to the curvature of the disc-shaped base 54 of the atom pieces when the chemical bond pieces are position between two atom pieces to represent the formation of a chemical bond. In a variation, each piece of the set of chemical pieces includes a magnetic 70 to magnetically attach to the target area when the target area also includes one or magnets. When the pieces include a magnet, the active area can be defined by a magnetic board (e.g., a magnetic whiteboard) that defines the target area. Although not limited by the dimensions, disc-shaped base can have a diameter from 15 mm to 60 mm and a thickness from about 2 to 5 mm. Similarly, elongated base section 64 can have a length from about 10 to 50 mm, a width from 5 to 20 mm, and a thickness from about 2 to 5 mm. The embodiments can extend from 1 mm to 5 mm from the disc-shaped base.

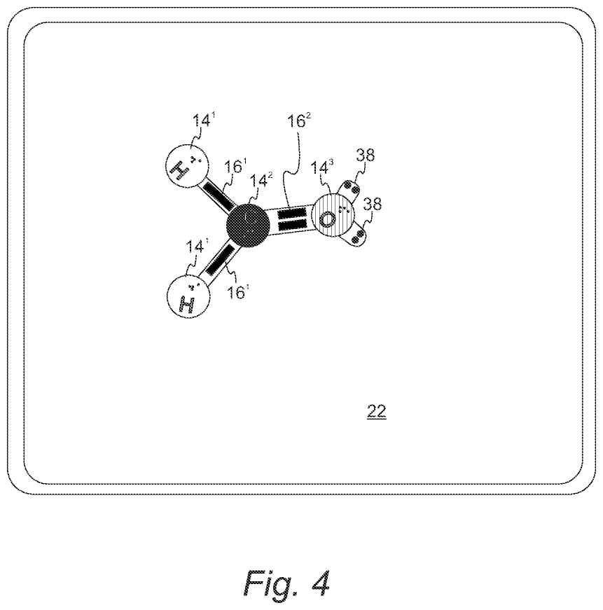

Referring to FIG. 4, a schematic illustrating the method for using the augmented reality Lewis structure explorer of FIG. 1A and 1B is provided. The user selects and places atom pieces 14 and chemical bond pieces onto active area 22 to create chemical molecules. Lone pair pieces 38 are placed on atom pieces as needed to complete the octet for each atom. Hint token 48 can be placed adjacent to each atom piece to provide feedback about the electronic state of that atom. Similarly, the set of chemical pieces can be used to construct representations of chemical reactions and/or chemical mechanisms.

In a variation, the computer device 32 is configured to recognize each member of the set of chemical pieces (e.g., atom piece and chemical bond pieces) by one or more computer vision algorithms. In particular, the computer device is configured to recognize each member of the set of chemical pieces (e.g., atom piece and chemical bond pieces) by shape recognition and color recognition. A useful software library for this recognition is the Open Source Computer Vision (“OpenCV”) Library which is a library of computer vision functions. In another variation, the computer device 32 is configured to recognize each member of the set of chemical pieces with a trained neural network. In this regard, an untrained neural network can be trained with a training set including pairs of a member of the set of chemical pieces and a label for that piece to form a trained neural network.

In a variation, computer device 32 is further configured to provide audio feedback and/or guidance to a user when a representation for molecule is formed with chemical pieces or when a representation for a chemical reaction or mechanism is constructed with the chemical pieces. This feedback and guidance can be provided with or without utilization of the hint token. Therefore, the system can be set to provide such feedback or/guidance automatically. For example, an audio feedback and/or guidance can be provided naming a representation compound is formed. In another refinement, the audio feedback and/or guidance can provide an indication whether or not a constructed reaction or mechanism is chemically balanced and/or suggestions for balancing the constructed chemical reaction representation.

In another variation, a chemical structure and/or a chemical mechanism system can include a chemical structure and/or a chemical mechanism program running a computer device such as computer device 32 with the augmented reality Lewis structure explorer functioning as an input device. An example of such a program is provided in International Pat. Pub. W02020180771A1 which was published on Sep. 10, 2020; the entire disclosure of this publication is incorporated herein in its entirety. In this example, a mechanisms authoring and data collection system includes an authoring tool, a user tool (e.g., used by a student), and optionally a monitoring tool (e.g., used by a teacher) as explained below in more detail. When applied to this example, the augmented reality Lewis structure explorer can be used to provide inputs to the user tool. In a refinement, a series of inputs from the user are received on a user tool on the user computer device for moving atoms and or bonds in the chemical rendering of the starting chemical compounds to reproduce a chemical mechanism. The authoring tool is used create a mechanisms problem to be solved by the user. The monitoring tool is used to monitor a user's progress in solving the mechanisms problem.

In another variation, the augmented reality Lewis structure explorer allows move-by-move data to be collected from a plurality of users using the augmented reality Lewis structure explorer as crowd source data. For example, move-by-move data is collected from a plurality of users in solving one or more Lewis Structure exercises. Moves made by the users can be characterized and stored as explained in International Pat. Pub. W02020180771A1 which was published on Sep. 10, 2020; the entire disclosure of this publication is incorporated herein. In a refinement, the moves are collected on a centralized computer device (e.g., a centralized server).

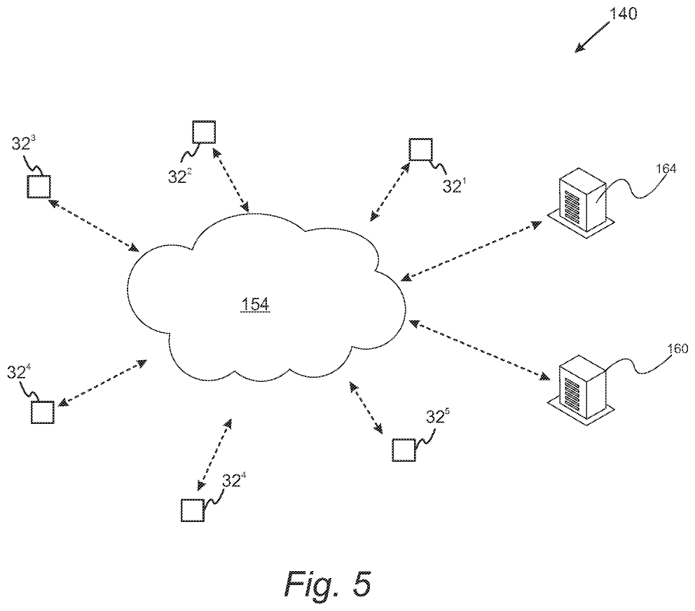

With reference to FIG. 5, a schematic of networked computer devices implementing the augmented reality Lewis structure explorer. Networked augmented reality Lewis structure explorer system 140 includes one or more user computer devices 32¹-32⁵ (e.g., 1 or 2 to 10,000 or more) communicating over network 154, The functionality of the user computer devices is set forth above. Network 154 can be a wired and/or wired network. Typically, network 154 will operate over the Internet. Networked mechanism teaching and data collection system 140 includes one or more monitoring tools 160 as set forth in International Pat. Pub. W02020180771A1. In a refinement, monitoring tool 160 store tracking data on database 162 either implemented on monitoring tool 160 or a centralized server 164.

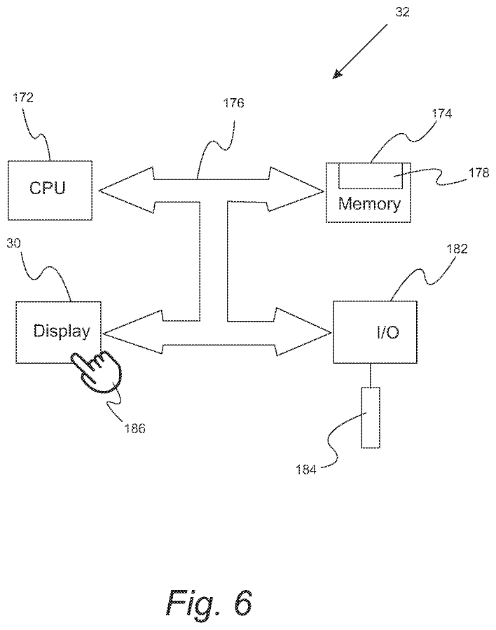

As set forth above, the augmented reality Lewis structure explorer includes a computer device. With reference to FIG. 6, computer device 32 includes computer processor 172 that executes the instructions for the augmented reality Lewis structure explorer. It should be appreciated that virtually a type of computer processor may be used, including microprocessors, multi-core processors, and the like. The instructions for the method typically are stored in computer memory 174 and accessed by computer processor 172 via connection system 176. In a variation, connection system 176 is and/or includes a data bus. In a refinement, computer memory 174 includes a computer-readable medium 178 which can be any non -transitory (e. g., tangible) medium that participates in providing data that may be read by a computer. Specific examples for computer memory 174 include, but are not limited to, random access memory (RAM), read-only memory (ROM), hard drives, optical drives, removable media (e.g., compact disks (CDs), DVD, flash drives, memory cards, etc.), and the like, and combinations thereof. In another refinement, computer processor 172 receives instructions from computer memory 174 and executes these instructions, thereby performing one or more processes, including one or more of the processes described herein. Computer-executable instructions may be compiled or interpreted from computer programs created using a variety of programming languages and/or technologies including, without limitation, and either alone or in combination, Java, C, C⁺⁺, C#, Fortran, Pascal, Visual Basie, Java Script Perl, PL/SQL, etc. Display 30 is also in communication with computer processor 172 via connection system 176. Computer device 32 also includes various in/out ports 182 through which data from a pointing device 184 may be accessed by computer processor 172. Examples for the computer device include, but are not limited to, desktop computers, laptops, smartphones, tablets, or tablet computers. Specifically, the methods can be implemented by iPad, iPod, and other tablets. Examples of pointing devices include a mouse, touch screen, stylus, trackball, joystick, or touchpad. In a particularly useful variation, the pointing device is incorporated into display 178 as a touch screen by which user 186 interacts with a finger. In a variation, a non-transitory storage medium or media as set forth above has encoded thereon instructions for the steps executed by the augmented reality Lewis structure explorer.

In a variation, augmented reality Lewis structure explorer is configured as a chemical structure and/or chemical mechanism system is provided. Referring to FIG. 1A, computer device 32 executes a chemical structure and/or a chemical mechanism program. The augmented reality Lewis structure explorer described above is advantageously used as the input device for the chemical structure and/or a chemical mechanism program. Therefore, computer device 32 includes a software driver for accepting such input. International Pat. Pub. W02020180771A1 provides an example of such a chemical structure and/or a chemical mechanism program in which a chemical mechanism problem is presented to a user to solve. In such programs the augmented reality Lewis structure explorer allows a user to construct molecules and allows manipulations thereof to depict a chemical mechanism. In a refinement, the created chemical structures and mechanism manipulations are reflected on display 178.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

What is claimed is:

1. An augmented reality Lewis structure explorer comprising:
a set of chemical pieces to be used to form representations of chemical structures, the set of chemical pieces including:
a first plurality of atom pieces, each atom piece representing an atom;
a second plurality of chemical bond pieces, each chemical bond piece representing a chemical bond;
one or more video cameras configured to view a target area;
a display; and
a computer device in communication with the one or more video cameras and the display, the computer device configured to recognize atom pieces and chemical bond pieces placed within the target area, the computer device configured to recognize chemical bonds when a selected chemical bond piece is placed between two atom pieces, the computer device further configured to render recognize atom pieces and chemical bond pieces on the display.

2. The augmented reality Lewis structure explorer of claim 1 wherein each atom piece and each chemical bond piece includes a magnetic to magnetically attach to the target area when the target area also includes one or magnets.

3. The augmented reality Lewis structure explorer of claim 2 further comprising a magnetic board that defines the target area.

4. The augmented reality Lewis structure explorer of claim 1, wherein the chemical bond pieces includes single bond pieces representing single bonds, double bond pieces representing double bonds, and triple bond pieces representing triple bonds.

5. The augmented reality Lewis structure explorer of claim 1, wherein the set of chemical pieces further includes positive charge pieces representing positive charges and negative charge pieces representing negative charges.

6. The augmented reality Lewis structure explorer of claim 1, wherein the set of chemical pieces further includes electron pair pieces representing a lone pair of electrons and single electron pieces representing a radical.

7. The augmented reality Lewis structure explorer of claim 1 wherein the set of chemical pieces further includes a hint token that provides feedback regarding the number of valence electrons and/or formal charge when the hint token is positioned proximate to an atom piece or a chemical bond piece.

8. The augmented reality Lewis structure explorer of claim 7, wherein the feedback includes an indication of missing parts of a chemical bond or octet.

9. The augmented reality Lewis structure explorer of claim 1, wherein the computer device is configured to recognize atom piece and chemical bond pieces by one or more computer vision algorithms.

10. The augmented reality Lewis structure explorer of claim 9, wherein the computer device is configured to recognize atom piece and chemical bond pieces by shape recognition and color recognition.

11. The augmented reality Lewis structure explorer of claim 1, wherein move-by-move data is collected from a plurality of users using the augmented reality Lewis structure explorer as crowd source data.

12. The augmented reality Lewis structure explorer of claim 1, wherein move-by-move data is collected from a plurality of users in solving one or more Lewis Structure exercises as crowd source data.

13. The augmented reality Lewis structure explorer of claim 1, wherein the computer device is configured to provide audio feedback and/or guidance to a user when a representation for molecule is formed with chemical pieces or when a representation for chemical reaction or mechanism is constructed with the set of chemical pieces.

14. A chemical structure and/or chemical mechanism system comprising a computer device running a chemical structure and/or chemical mechanism program;
and
an augmented reality Lewis structure explorer configured as an input device for the chemical structure and/or chemical mechanism program, the augmented reality Lewis structure explorer comprising:
a set of chemical pieces to be used to form representations of chemical structures, the set of chemical pieces including:
a first plurality of atom pieces, each atom piece representing an atom;
a second plurality of chemical bond pieces, each chemical bond piece representing a chemical bond; and
one or more video cameras configured to view a target area; and
a display, wherein the computer device is in communication with the one or more video cameras and the display, the computer device configured to recognize atom pieces and chemical bond pieces placed within the target area, the computer device configured to recognize chemical bonds when a selected chemical bond piece is placed between two atom pieces, the computer device further configured to render recognize atom pieces and chemical bond pieces on the display.

15. The chemical structure and/or chemical mechanism system of claim 14 wherein each atom piece and each chemical bond piece includes a magnetic to magnetically attach to the target area when the target area also includes one or magnets.

16. The chemical structure and/or chemical mechanism system of claim 15 further comprising a magnetic board that defines the target area.

17. The chemical structure and/or chemical mechanism system of claim 14, wherein the chemical bond pieces includes single bond pieces representing single bonds, double bond pieces representing double bonds, and triple bond pieces representing triple bonds.

18. The chemical structure and/or chemical mechanism system of claim 14, wherein the set of chemical pieces further includes positive charge pieces representing positive charges and negative charge pieces representing negative charges.

19. The chemical structure and/or chemical mechanism system of claim 14, wherein the set of chemical pieces further includes electron pair pieces representing a lone pair of electrons and single electron pieces representing a radical.

20. The chemical structure and/or chemical mechanism system of claim 14, wherein the computer device is configured to recognize atom piece and chemical bond pieces by one or more computer vision algorithms.

21. The chemical structure and/or chemical mechanism system of claim 20, wherein the computer device is configured to recognize atom piece and chemical bond pieces by shape recognition and color recognition.

22. The chemical structure and/or chemical mechanism system of claim 20, wherein the computer device is configured to provide audio feedback and/or guidance to a user when a representation for molecule is formed with chemical pieces or when a representation for chemical reaction or mechanism is constructed with the set of chemical pieces.
